(19) United States
(12) Patent Application Publication
(10) Pub. No.: US 2017/0143616 A1
(43) Pub. Date: May 25, 2017

(54) ANHYDROUS COMPOSITION INCLUDING A LIPOPHILIC GELLING AGENT, AT LEAST ONE SPECIFIC FILLER AND AN OIL PHASE

(71) Applicant: L'OREAL, Paris (FR)

(72) Inventors: Valerie PAGE, Saou (FR); Sophie GUILBAUD, Cliousclat (FR); Celine DEMARCQ, Piegros La Claster (FR)

(73) Assignee: L'OREAL, Paris (FR)

(21) Appl. No.: 15/322,488

(22) PCT Filed: Jun. 30, 2015
(86) PCT No.: PCT/FR2015/051799

§ 371 Date: Dec. 28, 2016

(30) Foreign Application Priority Data
Jun. 30, 2014 (FR) 1456211

(51) Int. Cl. A61K 8/92; A61K 8/60; A61K 8/37; A61Q 1/00; A61K 8/73; A61K 8/44; A61K 8/31; A61K 8/02; A61K 8/25

(52) U.S. Cl. CPC A61K 8/922; A61K 8/025; A61K 8/604; A61K 8/37; A61K 8/25; A61K 8/731; A61K 8/44; A61K 8/31; A61Q 1/00; A61K 2800/31

(57) ABSTRACT

The present invention relates to an anhydrous composition comprising:
from 3% to 15% by weight of at least one lipophilic gelling agent;
from 20% to 50% by weight of at least one filler chosen from spherical porous silica particles, spherical cellulose particles and powders of an N-acylamino acid bearing a C8-C22 acyl group; and
from 40% to 85% by weight of at least one fatty phase containing at least 20% by weight of at least one oil chosen from hydrocarbon-based oils of plant origin, preferably triglycerides, Guerbet alcohol derivatives, preferably Guerbet alcohols and esters of a fatty acid and of a Guerbet alcohol, fatty alcohol ethers comprising from 6 to 20 carbon atoms, and linear alkanes comprising from 7 to 17 carbon atoms;
   the weight amounts being given relative to the total weight of the composition.

The composition in accordance with the invention affords comfortable anhydrous care with a soft-focus effect, i.e. it can render the skin microrelief optically matt and/or smooth, fill wrinkles, hide skin imperfections and better reflect light, while at the same time giving a pleasant feel and a novel sensory effect, that of oily care that is not greasy or tacky, with powdery transformation especially when it is applied, and with a velvety skin finish, and which allows the skin to breathe.

The present invention relates to an anhydrous composition comprising from 3% to 15% by weight of at least one lipophilic gelling agent, from 20% to 50% by weight of at least one filler chosen from spherical porous silica particles, spherical cellulose particles and powders of an N-acylamino acid bearing a C8-C22 acyl group, and from 40% to 85% by weight of at least one fatty phase containing at least 20% by weight of at least one oil chosen from hydrocarbon-based oils of plant origin, preferably triglycerides, Guerbet alcohols and esters of a C8-C30 fatty acid and of a Guerbet alcohol, fatty alcohol ethers comprising from 6 to 20 carbon atoms, and linear C7-C17 alkanes. The invention relates in particular to the cosmetic field, and especially to the field of caring for and/or making up keratin materials, especially the skin, the lips, the hair or the nails, preferably the skin.

In the field of cosmetic compositions for caring for and/or making up the skin, it is known practice to use mineral or organic fillers with a soft-focus effect which absorb fat, give an astounding “photoshop” optical effect, and also a very soft feel, for making the skin matt, and/or for optically smoothing the microrelief, filling wrinkles, hiding skin imperfections and better reflecting light.

However, the use of these fillers is generally accompanied by a dry, rough feel, fluffing, white marks and a lack of comfort that are unacceptable to the user.

In order to overcome this problem and to afford comfortable care that has a soft-focus effect, oily presentation forms are currently proposed on the cosmetic market. However, these forms have the drawbacks associated with the presence of large contents of fatty phase in the composition, namely producing shiny skin and/or the sensation of greasy and/or tacky skin.

It is also known practice to use crosslinked silicones to satisfy this problem. The reason for this is that raw materials of this type allow a matt effect and a soft-focus effect to be combined, but have the drawback of being characterized by a relatively unpleasant hot, greasy feel, with a “mask” effect.

Thus, it is difficult to reconcile in the same composition opposing technical performance qualities, for instance a matt result (which may make the skin dry) and moisturization (which may make the skin shiny).

It thus remains difficult for a person skilled in the art to propose homogeneous compositions that are capable of affording an immediate visual result on the skin with a sensation of lightness and comfort on application, this expected immediate result preferentially being good covering of color imperfections and/or relief imperfections.

There thus remains a need for cosmetic compositions which can solve the technical problem of making the skin's microrelief matt and/or optically smooth, filling wrinkles, hiding skin imperfections and better reflecting light, while at the same time affording a pleasant feel especially when applied, and a very soft skin finish with no “mask” effect, and which allow the skin to breathe.

The Applicant has discovered that this need can be satisfied by combining, in an anhydrous composition comprising a suitably selected fatty phase, a lipophilic gelling agent and suitably selected fillers, in appropriate amounts.

This combination makes it possible to satisfy the problem mentioned above by proposing an oily presentation form endowed with a novel sensory effect both on application and on the skin finish, without neglecting the cosmetic qualities.

More specifically, one subject of the present invention is an anhydrous composition comprising:
from 3% to 15% by weight of at least one lipophilic gelling agent;
from 20% to 50% by weight of at least one filler chosen from spherical porous silica particles, spherical cellulose particles and powders of an N-acylamino acid bearing a C8-C22 acyl group; and
from 40% to 85% by weight of at least one fatty phase containing at least 20% by weight of at least one oil chosen from hydrocarbon-based oils of plant origin, preferably triglycerides, Guerbet alcohols, esters of a C8-C30 fatty acid and of a Guerbet alcohol, fatty alcohol ethers comprising from 6 to 20 carbon atoms, and linear C7-C17 alkanes;
   the weight amounts being given relative to the total weight of the composition.

According to a particular embodiment of the invention, the anhydrous composition comprises:
from 3% to 15% by weight of at least one lipophilic gelling agent;
from 20% to 50% by weight of at least one filler chosen from spherical porous silica particles, spherical cellulose particles and powders of an N-acylamino acid bearing a C8-C22 acyl group; and
from 40% to 85% by weight of at least one fatty phase containing at least 25% by weight of at least one oil chosen from hydrocarbon-based oils of plant origin, preferably triglycerides, Guerbet alcohols, esters of a C8-C30 fatty acid and of a Guerbet alcohol, and linear C7-C17 alkanes;
   the weight amounts being given relative to the total weight of the composition.

The present invention makes it possible to obtain a novel galenical form that is most particularly advantageous with regard to its technical performance qualities and the sensory results it affords the user when it is applied to keratin materials, and in particular to the skin.

Thus, the composition in accordance with the invention provides anhydrous comfortable care with a soft-focus effect, i.e. making it possible to make the skin's microrelief matt and/or optically smooth, to fill wrinkles, hide skin imperfections and better reflect light. It affords an optical and tactile effect for immediately correcting the surface appearance of the skin, by attenuating the shadow areas of the skin relief such as wrinkles, fine lines, dilated pores and other skin imperfections. It fades out the signs of fatigue. It smooths and refines the skin grain, restoring the skin's radiance and freshness, for a natural result.

The composition satisfies consumers' need for an oily product without having the application and skin finish drawbacks thereof generated by a conventional type of oily presentation form, namely without having a greasy feel or a shiny appearance, and being sparingly tacky or non-tacky. The composition penetrates well when it is applied to the skin.

The skin finish may be powdery, velvety, homogeneous and comfortable.

The skin finish may also be a film-forming finish that is advantageously coating, and sparingly tacky or non-tacky.

The comfort on application is reflected especially by an absence of tautness and of dryness sensations.

The composition according to the invention is stable, especially over time and/or with respect to temperature variations, while at the same time having good cosmetic and sensory properties.

The term “stable” means stable at room temperature (25° C.) for at least 1 month, preferably for at least 2 months.

Another subject of the present invention is a cosmetic process for making up and/or caring for keratin materials, comprising a step of applying a composition as defined above to said keratin materials.

As the composition of the invention is intended for topical application to the skin or the integuments, it comprises a physiologically acceptable medium, i.e. a medium that is compatible with all keratin materials, such as the skin, the nails, mucous membranes and keratin fibers (such as the hair or the eyelashes).

For the purposes of the present invention, the term “anhydrous” refers to a composition comprising a content of less than or equal to 1% by weight and preferably less than or equal to 0.5% by weight of water relative to the total weight of said composition, or is even free of water. Where appropriate, such small amounts of water may especially be introduced by ingredients of the composition that may contain residual amounts thereof.

In the text hereinbelow, the expression “at least one” is equivalent to “one or more” and, unless otherwise indicated, the limits of a range of values are included in that range.

### Lipophilic Gelling Agents

For the purposes of the present invention, the term “lipophilic gelling agent” means a compound that is capable of gelling the fatty phase of the composition according to the invention.

The lipophilic gelling agent(s) are liposoluble or lipodispersible.

According to a particular embodiment of the invention, the lipophilic gelling agent(s) are organic or mineral.

According to another particular embodiment of the invention, the lipophilic gelling agent(s) are non-silicone-based.

The gelling agent(s) that may be used in the context of the invention may be organic, polymeric or molecular lipophilic gelling agents.

As examples of lipophilic gelling agents that may be used in the context of the invention, mention may be made of modified natural micas such as aluminum magnesium potassium fluorosilicate, in particular the product sold by the company Sensient under the name Submica M, fatty acid esters of dextrin, especially of C7-C51, such as dextrin palmitate, in particular the product sold by the company Chiba Flour Milling under the name Rheopearl TL2-OR, or the dextrin palmitate sold by the same company under the name Rheopearl KL2-OR, and also dextrin myristate, in particular the product sold by the company Chiba Flour Milling under the name Rheopearl MKL2, triesters of a C8-C30 fatty acid and of mono- or polyglycerol, such as glyceryl trihydroxystearate, in particular the product sold by the company Elementis under the name Thixcin R. Mention may also be made of Rheocin sold by the company Byk Additives & Instruments.

According to a particular embodiment of the invention, the lipophilic gelling agent(s) are organic.

According to a particular embodiment, the lipophilic gelling agent(s) that may be used in the context of the invention are chosen from fatty acid esters of dextrin and triesters of a C8-C30 fatty acid and of mono- or polyglycerol.

### Fatty Acid Esters of Dextrin

The fatty acid esters of dextrin used according to the invention may be chosen especially from monoesters or polyesters of dextrin and of at least one fatty acid, corresponding to formula (C):

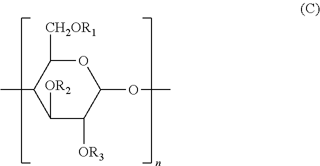

in which:
n is an integer ranging from 3 to 150, especially from 10 to 100 and preferably from 15 to 40; the radicals R₁, R₂ and R₃ represent a hydrogen atom or an acyl group (R—CO—) in which the radical R is a linear or branched, saturated or unsaturated hydrocarbon-based group containing from 6 to 50, especially from 8 to 30, or even 12 to 22 and better still 12 to 18 carbon atoms, with the proviso that at least one of said radicals R₁, R₂ or R₃ is other than a hydrogen atom.

In particular, R₁, R₂ and R₃ may represent hydrogen or an acyl group (R—CO—) in which R is a hydrocarbon-based radical as defined previously, with the proviso that at least two of said radicals R₁, R₂ or R₃ are identical and other than hydrogen.

The radicals R₁, R₂ and R₃ may all represent an acyl group (R—CO), which is identical or different and especially identical.

The radical R—CO— of the dextrin ester of formula (C) may be chosen especially from caprylyl, caproyl, lauroyl, myristyl, palmityl, stearyl, eicosanyl, docosanoyl, isovaleryl, 2-ethyl butyryl, ethylmethylacetyl, isoheptanyl, 2-ethylhexanyl, isononanyl, isodecanyl, isotridecanyl, isomyristyl, isopalmityl, isostearyl, isohexanyl, decenyl, dodecenyl, tetradecenyl, myristyl, hexadecenoyl, palmitoleyl, oleyl, elaidyl, eicosenyl, sorbyl, linoleyl, linolenyl, punicyl, arachidonyl and stearolyl radicals, and mixtures thereof.

The radical R—CO is advantageously linear. R—CO is preferably a palmityl radical or a myristyl radical, and even more preferentially a palmityl radical.

n advantageously ranges from 25 to 35, preferably from 27 to 33, and better is equal to 30.

Preferably, at least one dextrin palmitate and/or at least one dextrin myristate is used as fatty acid ester of dextrin. They may be used alone or as a mixture with other esters.

Advantageously, the fatty acid ester of dextrin has a degree of substitution of less than or equal to 2.5, especially ranging from 1.5 to 2.5, on the basis of one glucose unit. The weight-average molecular weight of the dextrin ester may in particular be from 10 000 to 150 000, especially from 12 000 to 100 000 and even from 15 000 to 80 000.

Dextrin esters are commercially available, in particular dextrin palmitates, for example under the name Rheopearl TL2-OR or Rheopearl KL2-OR from the company Chiba Flour Milling, and under the name Rheopearl KS from the company Chiba Flour Milling, and dextrin myristates, for example under the name Rheopearl MKL2 from the company Chiba Flour Milling.

According to a particular embodiment of the invention, use will be made of a mixture of a fatty acid ester of dextrin whose degree of substitution is less than 2 on the basis of one glucose unit and of a fatty acid ester of dextrin whose degree of substitution is greater than 2 on the basis of one glucose unit, as described in patent application FR 2 843 020.

According to one embodiment, the fatty acid ester of dextrin whose degree of substitution is less than 2 on the basis of one glucose unit advantageously corresponds to formula (IV) below:

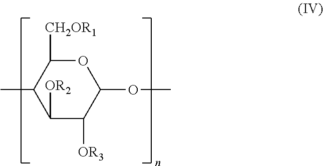

in which:
the radicals R₁, R₂ and R₃ represent a hydrogen atom or an acyl group (R—CO—) in which the radical R is a linear or branched, saturated or unsaturated hydrocarbon-based group containing from 6 to 50, especially from 8 to 30, or even 12 to 22 and better still 12 to 18 carbon atoms, with the proviso that at least one of said radicals R₁, R₂ or R₃ is other than a hydrogen atom.

n is an integer ranging from 3 to 150, especially from 10 to 100 and preferably from 15 to 40.

The radical R—CO— of the dextrin ester of formula (IV) may be chosen especially from caprylyl, caproyl, lauroyl, myristyl, palmityl, stearyl, eicosanyl, docosanoyl, isovaleryl, 2-ethylbutyryl, ethylmethylacetyl, isoheptanyl, 2-ethylhexanyl, isononanyl, isodecanyl, isotridecanyl, isomyristyl, isopalmityl, isostearyl, isohexanyl, decenyl, dodecenyl, tetradecenyl, myristyl, hexadecenoyl, palmitoleyl, oleyl, elaidyl, eicosenyl, sorbyl, linoleyl, linolenyl, punicyl, arachidonyl and stearolyl radicals, and mixtures thereof.

The radical R—CO is advantageously linear. The radical R—CO is preferably a palmityl radical or a myristyl radical, and even more preferentially a palmityl radical.

n advantageously ranges from 25 to 35, preferably from 27 to 33, and better is equal to 30.

Preferably, use is made of a fatty acid ester of dextrin whose degree of substitution is less than 2 on the basis of one glucose unit, such that the degree of substitution is less than 1.9, preferably less than 1.8 and more preferably is between 1.5 and 1.7. Some of these dextrin esters are commercially available, especially under the name Rheopearl TL from the company Chiba Flour Milling.

The weight-average molecular weight of the fatty acid ester of dextrin whose degree of substitution is less than 2 on the basis of one glucose unit is preferably between 10 000 and 30 000, more preferably between 15 000 and 20 000. The weight-average molecular weight is determined by gas chromatography, with polystyrene calibration.

According to one embodiment, the fatty acid ester of dextrin whose degree of substitution is greater than 2 on the basis of one glucose unit corresponds to formula (V):

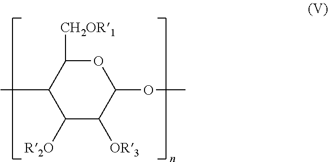

in which:
the radicals R′₁, R′₂ and R′₃, which may be identical or different, are chosen from a hydrogen atom and an acyl group (R′—CO—) in which the radical R′ is a linear or branched, saturated or unsaturated hydrocarbon-based group containing from 6 to 50, especially from 8 to 30, or even 12 to 22 and better still 12 to 18 carbon atoms, with the proviso that at least one of said radicals R′₁, R′₂ or R′₃ is other than a hydrogen atom;
n is an integer ranging from 3 to 150, especially from 10 to 100 and preferably from 15 to 40. R′ and n may have the same meaning as R and n described previously.

Advantageously, the radicals R′₁, R′₂ and R′₃ are identical, with the proviso that at least one of said radicals R′₁, R′₂ or R′₃ is other than a hydrogen atom.

Preferably, use is made of a fatty acid ester of dextrin whose degree of substitution is greater than 2 on the basis of one glucose unit, such that the degree of substitution is greater than 2.1, preferably between 2.1 and 2.3.

The weight-average molecular weight of the fatty acid ester of dextrin whose degree of substitution is greater than 2 on the basis of one glucose unit is preferably between 10 000 and 30 000, more preferably between 15 000 and 20 000. The weight-average molecular weight is determined by gas chromatography, with polystyrene calibration.

As examples of dextrin esters of formula (V) according to the invention, mention may be made of Rheopearl KL sold by the company Chiba Flour Milling.

### Triesters of Fatty Acid and of Mono- or Polyglycerol

According to a particular embodiment, the triester(s) of a fatty acid and of mono- or polyglycerol are triesters of fatty acid and of monoglycerol.

The term “fatty acid” means a linear or branched, saturated or unsaturated acid, comprising from 8 to 30 carbon atoms, preferably from 10 to 24 carbon atoms, even more preferentially from 12 to 22 and better still from 16 to 20 carbon atoms, optionally substituted with one or more hydroxyl groups. According to a particular embodiment of the invention, the fatty acid(s) are linear and saturated, substituted with at least one hydroxyl group.

The fatty acids may be chosen from oleic acid, lauric acid, palmitic acid, myristic acid, stearic acid, linoleic acid, capric acid and behenic acid, optionally substituted with at least one hydroxyl group, or mixtures thereof. Preferably, the fatty acid(s) are chosen from stearic acid, stearic acids substituted with at least one hydroxyl group, and mixtures thereof, and are more preferentially chosen from stearic acid, 12-hydroxystearic acid, and mixtures thereof, and even better still it is 12-hydroxystearic acid.

According to a particular embodiment of the invention, the triester of a C8-C30 fatty acid and of mono- or polyglyceryl is glyceryl tris(12-hydroxystearate).

As examples of triester of a C8-C30 fatty acid and of mono- or polyglycerol, mention may be made of glyceryl tri(hydroxystearate) (INCI name: Trihydroxystearin), for instance the product sold by the company Elementis under the name Thixcin R or the product sold by the company Byk Additives & Instruments under the name Rheocin.

According to a particular embodiment of the invention, the lipophilic gelling agent(s) that may be used in the context of the invention are chosen from fatty acid esters of dextrin, preferably dextrin palmitate and dextrin myristate.

According to another particular embodiment of the invention, the lipophilic gelling agent(s) that may be used in the context of the invention are chosen from triesters of a C8-C30 fatty acid and of monoglycerol, preferably glyceryl tri(hydroxystearate), and even more preferentially glyceryl tris(12-hydroxystearate).

The lipophilic gelling agent(s) are present in the composition in accordance with the invention in an amount of between 3% and 15% by weight, preferably from 5% to 12% by weight and even more preferentially from 8.5% to 10% by weight, relative to the total weight of the composition.

### Fillers

The composition in accordance with the invention comprises from 20% to 50% by weight, relative to the total weight of the composition, of at least one filler chosen from spherical porous silica particles, spherical cellulose particles and powders of an N-acylamino acid bearing a C8-C22 acyl group.

The term “filler” should be understood as meaning colorless or white, mineral or synthetic particles of any shape, which are insoluble in the medium of the composition, irrespective of the temperature at which the composition is manufactured.

The fillers used in the present invention may be characterized by their specific surface area per unit mass or per unit volume, their size expressed as the volume-mean diameter D(4,3), their non-tapped density and/or their oil-absorbing capacity.

The specific surface per unit of weight can be determined by the nitrogen absorption method, known as the BET (Brunauer-Emmett-Teller) method, described in The Journal of the American Chemical Society, Vol. 60, page 309, February 1938, which corresponds to International Standard ISO 5794/1 (Appendix D). The BET specific surface corresponds to the total specific surface of the particles under consideration.

The specific surface area per unit of volume is given by the relationship: S_{V}=S_{M}×p, where p is the tapped density, expressed in g/cm³, and S_{M} is the specific surface area per unit mass, expressed in m²/g, as defined above.

In the context of the present invention, this density, known as the tapped density, may be assessed according to the following protocol:

40 g of powder are poured into a measuring cylinder; the measuring cylinder is then placed on a Stay 2003 machine from Stampf Volumeter; the measuring cylinder is then subjected to a series of 2500 tapping actions (this operation is repeated until the difference in volume between two consecutive tests is less than 2%); the final volume Vf of tapped powder is then measured directly on the measuring cylinder. The tapped density is determined by the ratio m/Vf, in this instance 40/Vf (Vf being expressed in cm³ and m in g).

The oil-absorbing capacity, measured at the wet point and denoted Wp, corresponds to the amount of oil which it is necessary to add to 100 g of particles in order to obtain a homogeneous paste.

It is measured according to the “wet point” method or the method for determining the oil uptake of a powder described in standard NF T 30-022. It corresponds to the amount of oil adsorbed onto the available surface of the powder and/or absorbed by the powder by measuring the wet point, described below:

An amount m=2 g of powder is placed on a glass plate, and the oil (isononyl isononanoate) is then added dropwise. After addition of 4 to 5 drops of oil to the powder, mixing is performed using a spatula, and addition of oil is continued until conglomerates of oil and powder have formed. From this point, the oil is added one drop at a time and the mixture is then triturated with the spatula. The addition of oil is stopped when a firm, smooth paste is obtained. This paste must be able to be spread over the glass plate without cracks or the formation of lumps. The volume Vs (expressed in ml) of oil used is then noted.

The oil uptake corresponds to the ratio Vs/m.

According to a particular embodiment, the fillers that may be used in the present invention have an oil-absorbing capacity of from 0.25 to 3.5 mL/g, preferably from 0.93 to 2.5 mL/g, or even from 1.25 to 2.5 mL/g.

The sizes of the fillers may be measured by static light scattering using a commercial particle size analyzer such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is in particular described in the publication by Van de Hulst, H.C., Light Scattering by Small Particles, Chapters 9 and 10, Wiley, New York, 1957.

According to a particular embodiment, the fillers that may be used in the present invention have a size, expressed as the volume-mean diameter D(4,3), ranging from 0.1 μm to 40 μm, preferably from 0.5 μm to 20 μm and even more preferentially from 1 μm to 16 μm. According to a particular embodiment, the fillers used in the present invention have a non-tapped density ranging from 0.2 g/cm³ to 2.2 g/cm³.

According to a particular embodiment, they have a specific surface area ranging from 30 m²/g to 1000 m²/g and more particularly from 150 m²/g to 800 m²/g.

In the present patent application, the term “spherical particles” means particles in the form or substantially in the form of a sphere, which are insoluble in the medium of the composition according to the invention, even at the melting point of the medium (about 100° C.).

According to a particular embodiment of the invention, the spherical porous silica particles are microparticles. Preferably, they have a size, expressed as the volume-mean diameter D(4,3), ranging from 0.5 to 30 μm, more particularly from 1 to 20 μm and preferentially from 1 to 16 μm.

As examples of porous silica microbeads, use may be made of the following commercial products: Silica Beads SB-150, SB-300 or SB 700, preferentially SB 300 from the company Miyoshi Kasei; the Sunsphere range from the company Asahi Glass AGC SI-TECH, especially Sunsphere H-51 or Sunsphere 12L, Sunsphere H-201 H-52 and H-53; Sunsil 130 from the company Sunjin; Spherica P-1500 from the company Ikeda Corporation; Sylosphere from the company Fuji Silysia; the Silica Pearl and Satinier ranges from the company JGC Catalysts and Chemicals, more particularly Satinier M13 and M16, the silicas MSS-500 from the company Kobo, and more particularly MSS-500-20N, and also Silica Shells from the company Kobo.

According to a particular embodiment, the spherical cellulose particles that may be used in the context of the invention are microparticles. Preferably, they have a size, expressed as the volume-mean diameter D(4,3), ranging from 0.1 to 35 μm, preferably from 1 to 20 μm and more particularly from 4 to 15 μm.

Examples of spherical cellulose microparticles that may especially be mentioned include the solid cellulose beads sold under the names Cellulobeads D-10, Cellulobeads D-5 and Cellulobeads USF by the company Daito Kasei Kogyo.

The N-acylamino acids comprise an acyl group containing from 8 to 22 carbon atoms, for instance a 2-ethylhexanoyl, caproyl, lauroyl, myristoyl, palmitoyl, stearoyl or cocoyl group.

The amino acid may be, for example, lysine, glutamic acid or alanine, preferably lysine.

According to a particular embodiment, the N-acylamino acid(s) comprise an acyl group containing from 10 to 14 carbon atoms. Preferably, it is a lauroyl group. Advantageously, the N-acylamino acid powder may be a lauroyllysine powder such as the product sold under the name Amihope LL by the company Ajinomoto or the product sold under the name Corum 5105 S by the company Corum.

According to a particular embodiment of the invention, the fillers chosen from spherical porous silica particles, spherical cellulose particles and powders of an N-acylamino acid bearing a C8-C22 acyl group have a heterogeneous particle size, i.e. a large size distribution for a given size expressed as the volume-mean diameter.

The composition in accordance with the invention comprises from 20% to 50% by weight and preferably from 25% to 35% by weight, relative to the total weight of the composition, of at least one filler chosen from spherical porous silica particles, spherical cellulose particles and powders of an N-acylamino acid bearing a C8-C22 acyl group.

According to one embodiment in accordance with the invention, the composition comprises from 20% to 50% by weight and preferably from 25% to 35% by weight, relative to the total weight of the composition, of a single filler chosen from spherical porous silica particles, spherical cellulose particles and powders of an N-acylamino acid bearing a C8-C22 acyl group.

According to a first particular embodiment, the composition in accordance with the invention comprises from 25% to 50% by weight and preferably from 25% to 35% by weight, relative to the total weight of the composition, of at least one filler chosen from spherical porous silica particles. Advantageously, the composition in accordance with the invention may comprise from 25% to 50% by weight and preferably from 25% to 35% by weight, relative to the total weight of the composition, of a single filler chosen from spherical porous silica particles.

According to a second particular embodiment, the composition in accordance with the invention comprises from 25% to 50% by weight and preferably from 25% to 35% by weight, relative to the total weight of the composition, of at least one filler chosen from spherical cellulose particles. Advantageously, the composition in accordance with the invention may comprise from 25% to 50% by weight and preferably from 25% to 35% by weight, relative to the total weight of the composition, of a single filler chosen from spherical cellulose particles.

According to a third particular embodiment, the composition in accordance with the invention comprises from 25% to 50% by weight and preferably from 25% to 35% by weight, relative to the total weight of the composition, of at least one filler chosen from powders of an N-acylamino acid bearing a C8-C22 acyl group. Advantageously, the composition in accordance with the invention may comprise from 25% to 50% by weight and preferably from 25% to 35% by weight, relative to the total weight of the composition, of a single filler chosen from powders of an N-acylamino acid bearing a C8-C22 acyl group.

According to a fourth embodiment, the composition in accordance with the invention comprises at least two fillers chosen from spherical porous silica particles, spherical cellulose particles and powders of an N-acylamino acid bearing a C8-C22 acyl group.

According to a specific embodiment, the composition according to the invention comprises at least two fillers that are different from each other, one of which is chosen from porous spherical silica particles, in particular porous spherical silica microparticles, and the other is chosen from spherical cellulose particles and powders of an N-acylamino acid bearing a C8-C22 acyl group.

In particular, the composition may comprise at least two fillers that are different from each other, one of which is chosen from spherical cellulose particles and the other is chosen from porous spherical silica particles, in particular porous spherical silica microparticles.

The composition may also comprise at least two fillers that are different from each other, one of which is chosen from powders of an N-acylamino acid bearing a C8-C22 acyl group and the other is chosen from porous spherical silica particles, in particular porous spherical silica microparticles.

According to a fifth particular embodiment, the composition in accordance with the invention comprises at least three fillers chosen from spherical porous silica particles, spherical cellulose particles and powders of an N-acylamino acid bearing a C8-C22 acyl group.

According to a specific embodiment, the composition in accordance with the invention comprises at least three fillers that are different from each other, the first of which is chosen from porous spherical silica particles, in particular porous spherical silica microparticles, the second is chosen from spherical cellulose particles, and the third is chosen from powders of an N-acylamino acid bearing a C8-C22 acyl group.

According to a particular embodiment, the composition does not comprise any additional filler other than the spherical porous silica particles, the spherical cellulose particles and the powders of an N-acylamino acid bearing a C8-C22 acyl group.

Alternatively, the composition in accordance with the invention may also comprise at least one additional filler other than the spherical porous silica particles, the spherical cellulose particles and the powders of an N-acylamino acid bearing a C8-C22 acyl group.

The additional filler(s) may especially be organic fillers and/or mineral fillers.

The additional filler(s) may also be characterized by their specific surface area per unit mass or per unit volume, their size expressed as the volume-mean diameter D(4,3), their non-tapped density and/or their oil-absorbing capacity. The definition and the methods for measuring these parameters were described previously for the fillers chosen from spherical porous silica particles, spherical cellulose particles and powders of an N-acylamino acid bearing a C8-C22 acyl group.

According to a particular embodiment, the additional filler(s) that may be used in the present invention have an oil-absorbing capacity of from 0.25 mL/g to 3.5 mL/g, preferably from 0.93 mUg to 2.5 mL/g, or even from 1.25 mL/g to 2.5 mL/g.

According to a particular embodiment, the additional filler(s) that may be used in the present invention have a size, expressed as the volume-mean diameter D(4,3), ranging from 0.1 μm to 40 μm, preferably from 0.5 μm to 20 μm and even more preferentially from 1 μm to 16 μm.

According to a particular embodiment, the additional filler(s) that may be used in the present invention have a non-tapped density ranging from 0.2 g/cm³ to 2.2 g/cm³.

### Organic Fillers

In the present patent application, the term “organic filler” means any organic solid that is insoluble in the medium at room temperature (25° C.).

The term “organic” refers to any compound or polymer whose chemical structure comprises at least one or more carbon atoms.

As organic fillers that may be used in the composition of the invention, examples that may be mentioned include polyamide (Nylon®) particles and especially those sold under the name Orgasol® by the company Atochem; polyethylene powders; microspheres based on acrylic copolymers, such as those made of ethylene glycol dimethacrylate/lauryl methacrylate copolymer sold by the company Dow Corning under the name Polytrap®; polymethyl methacrylate microspheres, sold under the name Microsphere M-100® by the company Matsumoto or under the name Covabead LH85® by the company Wackherr; ethylene-acrylate copolymer powders, such as those sold under the name Flobeads® by the company Sumitomo Seika Chemicals; expanded powders such as hollow microspheres and especially microspheres formed from a terpolymer of vinylidene chloride, acrylonitrile and methacrylate and sold under the name Expancel® by the company Kemanord Plast under the references 551 DE 12® (particle size of about 12 μm), 551 DE 20® (particle size of about 30 μm), 551 DE 50® (particle size of about 40 μm), or the microspheres sold under the name Micropearl F 80 ED® by the company Matsumoto; powders of natural organic materials such as polysaccharide powders, and in particular starch powders, especially of crosslinked or non-crosslinked corn, wheat or rice starch, powders of starch crosslinked with octenylsuccinic anhydride, sold under the name Dry-Flo® by the company National Starch, powders of waxy corn starch, such as those sold under the names C* Gel 04201 by the company Cargill, Amidon de MaIs B by the company Roquette, and Organic Corn Starch by the company Draco Natural Products; silicone resin microbeads such as those sold under the name Tospearl by the company Toshiba Silicone, especially Tospearl 240; particles of wax microdispersion, which preferably have mean sizes of less than 25 μm and especially ranging from 0.5 μm to 25 μm, and which are formed essentially from a wax or a mixture of waxes, such as the products sold under the name Aquacer by the company Byk Cera, and especially: Aquacer 520 (mixture of synthetic and natural waxes), Aquacer 514® or 513® (polyethylene wax), Aquacer 511® (polymeric wax), or such as the products sold under the name Jonwax 120 by the company Johnson Polymer (mixture of polyethylene wax and paraffin wax) and under the name Ceraflour 961® by the company Byk Cera (micronized modified polyethylene wax); and mixtures thereof.

### Mineral Fillers

In the present patent application, the term “mineral filler” means any mineral solid that is insoluble in the medium at room temperature (25° C.).

The term “mineral” refers to any compound or polymer whose chemical structure does not comprise any carbon atoms.

As examples of additional mineral fillers, mention may be made of zeolites, such as the products sold by the company Zeochem under the names Zeoflair 300, Zeoflair 200, Zeoflair 100, X-Mol and X-Mol MT.

A zeolite is a crystal formed from an aluminosilicate microporous backbone, the connected empty spaces of which are initially occupied by cations and water molecules. They are also referred to as molecular sieves.

Mention may also be made of calcium magnesium carbonate, such as the products sold by the company Imerys under the name Calcidol, by the company LCW (Sensient) under the name Carbomat or by the company Omya under the name Omyacare S 60-AV.

Mention may also be made of lamellar mineral particles, such as talcs, micas or nacres, and mixtures thereof.

Talcs are hydrated magnesium silicates usually comprising aluminum silicate. The crystal structure of talc consists of repeated layers of a sandwich of brucite between layers of silica.

More particularly, the lamellar particles are chosen from talcs.

Advantageously, use is more particularly made, in the composition of the invention, as lamellar particles, of talc, such as the products sold under the names Luzenac Pharma M and UM by the company Imerys, Rose Talc and Talc SG-2000 by the company Nippon Talc; mica, such as the products sold under the names Mica M RP and Silk Mica by the company Merck; titanium micas such as mica/titanium oxide/brown iron oxide (CTFA: Mica/Iron oxides/Titanium dioxide), sold under the name Cloisonne Rouge Flambe 440 X by the company Engelhard. A mica that may be mentioned is the mica sold under the name Sericite S-152-BC by the company Miyoshi Kasei.

Among the mineral fillers, mention may be made of perlite particles and preferably expanded perlite particles.

The perlites that may be used according to the invention are generally aluminosilicates of volcanic origin and have as the composition:

70.0-75.0% by weight of silica SiO₂;
12.0-15.0% by weight of aluminum oxide A1₂0₃
3.0-5.0% of sodium oxide Na₂O;
3.0-5.0% of potassium oxide K₂O;
0.5-2% of iron oxide Fe₂O₃;
0.2-0.7% of magnesium oxide MgO;
0.5-1.5% of calcium oxide CaO;
0.05-0.15% of titanium oxide TiO₂.

Mention may be made especially of the perlites sold under the names Optimat 2550 OR by the company World Minerals, and Europerl EMP-2 and Europerl 1 by the company Imerys.

When the composition in accordance with the invention comprises at least one additional filler other than fillers chosen from spherical porous silica particles, spherical cellulose particles and powders of an N-acylamino acid bearing a C8-C22 acyl group, they are preferably present in an amount at least equal to 0.1% by weight and even more preferentially between 5% to 25% by weight relative to the total weight of the composition.

According to a particular embodiment, the content of additional fillers, when they are present, ranges from 0.1% to 10% by weight, preferably from 0.1% to 5% by weight, relative to the total weight of the composition.

According to a particular embodiment, when the composition comprises at least two different fillers, one of which is chosen from spherical porous silica particles, the mass ratio R (spherical porous silica particles)/(other fillers) is greater than or equal to 0.75, preferably between 0.75 and 3.

### Fatty Phase

The composition in accordance with the invention comprises from 40% to 85% by weight of a fatty phase, relative to the total weight of the composition.

According to a particular embodiment, the composition in accordance with the invention comprises from 45% to 85% by weight of a fatty phase. Preferably, the proportion of the fatty phase ranges from 45% to 75% by weight and even more preferentially from 57% to 70% by weight relative to the total weight of the composition.

This indicated amount does not comprise the content of lipophilic gelling agents such as those described above.

For the purpose of the invention, the fatty phase includes any fatty substance that is liquid at room temperature and atmospheric pressure, generally oils, or that is solid at room temperature and atmospheric pressure, like waxes, or any pasty compound, which are present in said composition.

The fatty substance(s) present in the composition may be chosen by a person skilled in the art on the basis of his general knowledge, so as to give the final composition the desired properties, especially in terms of rheological properties (penetrometry measurement, flow, consistency) or in terms of texture and stability.

The fatty phase of the composition in accordance with the invention comprises at least 20% by weight of at least one oil chosen from hydrocarbon-based oils of plant origin, preferably triglycerides, Guerbet alcohols, esters of a C8-C30 fatty acid and of a Guerbet alcohol, fatty alcohol ethers comprising from 6 to 20 carbon atoms, and linear C7-C17 alkanes.

According to a particular embodiment, the fatty phase of the composition in accordance with the invention comprises at least 25% by weight of at least one oil chosen from hydrocarbon-based oils of plant origin, preferably triglycerides, Guerbet alcohols, esters of a C8-C30 fatty acid and of a Guerbet alcohol, and linear C7-C17 alkanes.

According to another particular embodiment, the fatty phase of the composition in accordance with the invention comprises at least 25% by weight of at least two oils chosen from hydrocarbon-based oils of plant origin, preferably triglycerides, Guerbet alcohols, esters of a C8-C30 fatty acid and of a Guerbet alcohol, and linear C7-C17 alkanes.

The term “oils” means fatty substances that are liquid at room temperature (25° C.) and atmospheric pressure.

As hydrocarbon-based oils of plant origin, mention may be made of squalane, liquid triglycerides of fatty acids comprising from 4 to 30 carbon atoms, for instance heptanoic or octanoic acid triglycerides, or alternatively, for example, jojoba oil, babassu oil, sunflower oil, olive oil, coconut oil, Brazil nut oil, marula oil, corn oil, soybean oil, marrow oil, grapeseed oil, linseed oil, sesame seed oil, hazelnut oil, apricot oil, macadamia nut oil, arara oil, coriander oil, castor oil, avocado oil, caprylic/capric acid triglycerides, for instance those sold by the company Stéarineries Dubois or those sold under the names Miglyol 810, 812 and 818 by the company Dynamit Nobel, and shea butter oil.

According to a particular embodiment, the volatile linear alkanes, advantageously of plant origin, comprise from 7 to 17 carbon atoms, in particular from 9 to 15 carbon atoms and more particularly from 11 to 13 carbon atoms.

As examples of volatile linear alkanes that are suitable for use in the invention, mention may be made of those described in patent application WO 2007/068 371 from the company Cognis.

As example of a volatile linear alkane that is suitable for use in the invention, mention may be made of: n-nonane (C₉), n-decane (C₁₀), n-undecane (C₁₁), n-dodecane (C₁₂), n-tridecane (C₁₃), n-tetradecane (C₁₄), n-pentadecane (C₁₅), n-hexadecane (C₁₆) and n-heptadecane (C₁₇), and mixtures thereof. According to a particularly preferred embodiment, use will be made of a mixture of undecane (C₁₁) and of tridecane (0₁₃) such as the product sold under the reference Cetiol UT by the company Cognis. Mention may also be made of n-dodecane (C₁₂) and n-tetradecane (C₁₄) such as those sold by Sasol under the references, respectively, Parafol 12-97 and Parafol 14-97, and also mixtures thereof.

Use may also be made of Guerbet alcohols or of esters of C8-C30 fatty acids and of Guerbet alcohols. Guerbet alcohols are obtained by converting an aliphatic primary alcohol into a beta-alkylenated alcohol dimer via the following chemical reaction:

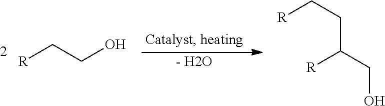

This reaction requires the presence of a base such as alkali metal hydroxides or alkali metal alkoxides, a catalyst such as Raney nickel, and high temperatures.

As Guerbet alcohols or esters of C8-C30 fatty acids and of Guerbet alcohols, mention may be made especially of octyldodecanol and octyldodecanol esters of a C8-C30 fatty acid, such as octyldodecyl myristate. Mention may be made in particular of octyldodecanol, such as the product sold under the name Eutanol G by the company Cognis (BASF) and the octyldodecyl myristate sold under the name Dub MOD by the company Gattefossé.

The fatty alcohol ethers that may be used in the context of the invention are ethers of fatty alcohols comprising from 6 to 20, preferably from 8 to 12 and even more preferentially from 8 to 10 carbon atoms.

These ethers may be obtained from two different fatty alcohols or from two identical fatty alcohols. Preferably, they are obtained from two identical fatty alcohols such as capryl alcohol (also known as 1-octanol or n-octanol). The corresponding ether is then dicaprylyl ether, such as the product sold under the name Cetiol OE by the company Cognis.

### Additional Oils

According to a particular embodiment, the composition according to the invention comprises at least one additional oil other than the oils chosen from hydrocarbon-based oils of plant origin, Guerbet alcohols, esters of a C8-C30 fatty acid and of a Guerbet alcohol, and linear C7-C17 alkanes.

As oils that may be used in the composition of the invention, examples that may be mentioned include:
synthetic esters and ethers, especially of fatty acids and/or of fatty alcohols, for instance the oils of formulae R¹COOR² and R¹OR² in which R¹ represents a fatty acid residue or R¹ represents a fatty alcohol residue containing from 8 to 29 carbon atoms and R² represents a branched or unbranched hydrocarbon-based chain containing from 3 to 30 carbon atoms, for instance purcellin oil, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate, and fatty alcohol heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;
mixtures thereof.

Mention may also be made of the following oils:
esters derived from the reaction of at least one fatty acid containing at least 6 carbon atoms, preferably from 6 to 26 carbon atoms, better still from 6 to 20 carbon atoms and even better still from 6 to 16 carbon atoms, and of at least one alcohol comprising from 1 to 17 carbon atoms and better still from 3 to 15 carbon atoms; mention may in particular be made of isopropyl myristate, such as the products sold under the name Palmester 1510 by the company KLK Oleo, under the name Lexol IPM-NF by the company Inolex Chemical Company or under the name Isopropyl Myristate by the company Cognis (BASF), isopropyl isostearate such as the product sold under the name Radia 7739 by the company Oleon, isopropyl palmitate, 2-ethylhexyl caprate/caprylate (or octyl caprate/caprylate), 2-ethylhexyl palmitate, isostearyl neopentanoate, isononyl isononanoate, hexyl laurate, lactic acid esters of fatty alcohols comprising 12 or 13 carbon atoms, and dicaprylyl carbonate, such as the product which is sold under the name Cetiol CC by the company Cognis,
glycerol ethers comprising from 6 to 12 carbon atoms, for instance the 2-ethylhexyl ether of glycerol (INCI name: ethylhexyl glycerol) such as Sensiva SC 50 from the company Schulke & Mayr GmbH.

Mention may be made in particular of the mixture of esters of caprylic/capric acids and of C12-C18 fatty alcohols such as coco-caprylate/caprate sold under the name Cetiol LC by the company Cognis or under the name Dub 810 C by the company Stéarineries Dubois;
polyesters obtained by condensation of a dimer and/or trimer of a C8-C30 unsaturated fatty acid and of diol, for instance the polyesters of dilinoleic acid and of diol sold by Biosynthis under the name Viscoplast and especially the polymer bearing the INCI name dilinoleic acid/propanediol copolymer;
and mixtures thereof.

The composition according to the invention may have a total oil content ranging from 40% to 85% by weight, preferably from 45% to 75% by weight and better still from 57% to 70% relative to the total weight of the composition.

The composition in accordance with the invention may also comprise at least one solid fatty substance such as pasty compounds and waxes.

### Pasty Compound

For the purposes of the present invention, the term “pasty compound” means a lipophilic fatty compound with a reversible solid/liquid change of state, and comprising at a temperature of 23° C. a liquid fraction and a solid fraction.

A pasty compound is, at a temperature of 23° C., in the form of a liquid fraction and a solid fraction. In other words, the starting melting point of the pasty compound is less than 23° C. The liquid fraction of the pasty compound, measured at 23° C., represents from 20% to 97% by weight of the pasty compound. This fraction that is liquid at 23° C. more preferentially represents from 25% to 85% and better still from 30% to 60% by weight of the pasty compound.

The liquid fraction by weight of the pasty compound at 23° C. is equal to the ratio of the heat of fusion consumed at 23° C. to the heat of fusion of the pasty compound. The heat of fusion consumed at 23° C. is the amount of energy absorbed by the sample to change from the solid state to the state that it has at 23° C., formed from a liquid fraction and a solid fraction.

The heat of fusion of the pasty compound is the heat consumed by the compound in order to pass from the solid state to the liquid state. The pasty compound is said to be in the solid state when all of its mass is in solid form. The pasty compound is said to be in the liquid state when all of its mass is in the liquid form.

The heat of fusion of the pasty compound is equal to the area under the curve of the thermogram obtained using a differential scanning calorimeter (DSC), such as the calorimeter sold under the name MDSC 2920 by TA Instruments, with a temperature rise of 5° C. or 10° C. per minute, according to Standard ISO 11357-3:1999. The heat of fusion of the pasty compound is the amount of energy required to make the compound change from the solid state to the liquid state. It is expressed in J/g.

The liquid fraction of the pasty compound measured at 32° C. preferably represents from 40% to 100% by weight of the pasty compound and better still from 50% to 100% by weight of the pasty compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the heat of fusion consumed at 32° C. to the heat of fusion of the pasty compound. The heat of fusion consumed at 32° C. is calculated in the same way as the heat of fusion consumed at 23° C.

The pasty compound preferably has a hardness at 20° C. ranging from 0.001 to 0.5 MPa and preferably from 0.002 to 0.4 MPa.

The hardness is measured according to a method of penetration of a probe into a sample of compound and in particular using a texture analyzer (for example the TA-XT2i machine from Rheo) equipped with a stainless-steel cylinder 2 mm in diameter. The hardness measurement is performed at 20° C. at the center of five samples. The cylinder is introduced into each sample, the penetration depth being 0.3 mm. The recorded hardness value is that of the maximum peak.

The pasty compound is chosen from compounds of plant origin. A pasty compound may be obtained via synthesis from starting materials of plant origin.

The pasty compound may be chosen especially from isomerized jojoba oil such as the trans-isomerized partially hydrogenated jojoba oil manufactured or sold by the company Desert Whale under the commercial reference Iso-Jojoba-50®, orange wax, for instance the product sold under the reference Orange Peel Wax by the company Koster Keunen, cupuacu butter (Rain Forest RF3410 from the company Beraca Sabara), murumuru butter (Rain Forest RF 3710 from the company Beraca Sabara), shea butter, partially hydrogenated olive oil, for instance the compound sold under the reference Beurrolive by the company Soliance, cocoa butter, mango oil, for instance Lipex 203 from the company Aarhuskarlshamn, and mixtures thereof.

Mention may also be made of mixtures of C8-C30 fatty acids and of C8-C30 fatty alcohols, such as mixtures of lauryl alcohol and methyl laurate, of stearyl alcohol and methyl palmitate or methyl behenate, such as the Purester range sold by the company Strahl & Pitsch, especially the lauryl laurate known under the trade name Purester 24.

Similarly, mention may be made of esters of a C8-C30 fatty acid and of polyglycerol comprising from 2 to 10 glyceryl units, such as the polyglyceryl-3 polyricinoleate sold by the company Aarhuskarlshamn under the name Akoline PGPR or alternatively a mixture of three jojoba ester waxes & Helianthus annus seed wax & Acacia decurrens extract and polyglyceryl, such as the product sold under the name Hydracire S or Acticire® by the company Gattefossé.

Mention may also be made of hydrogenated glycerol esters, for instance the product sold under the name Cegesoft HF 52 by the company Cognis (BASF), which is a mixture of hydrogenated rapeseed and palm oils, or the product sold under the name Softisan 100 Cremer by the company Oleo.

Mention may also be made of mixtures of monoester and/or diester of a C8-C18 fatty acid and of mono- or polyglycerol, such as glyceryl stearate, for instance the glyceryl stearate sold under the reference Cutina GMS V by the company Cognis or the mixture of glyceryl monostearate and distearate sold by the company Stéarineries Dubois under the name Dub GMS 50/50.

When they are present, the amount of pasty compounds may range, for example, from 0.05% to 85% by weight, better still from 0.1% to 40% by weight and in particular from 0.5% to 10% by weight relative to the total weight of the composition.

### Waxes

For the purposes of the present invention, the term “wax” means a lipophilic compound, which is solid at room temperature (25° C.), with a reversible solid/liquid change of state, having a melting point of greater than or equal to 30° C., which may be up to 120° C. In particular, the waxes have a melting point of greater than 30° C. and better still greater than 45° C.

For the purposes of the invention, the melting point corresponds to the temperature of the most endothermic peak observed on thermal analysis (differential scanning calorimetry or DSC) as described in standard ISO 11357-3; 1999. The melting point of the wax may be measured using a differential scanning calorimeter (DSC), for example the calorimeter sold under the name M DSC 2920 by the company TA instruments.

The measurement protocol is as follows:

A sample of 5 mg of wax placed in a crucible is subjected to a first temperature rise ranging from −20° C. to 100° C., at a heating rate of 10° C./minute, it is then cooled from 100° C. to −20° C. at a cooling rate of 10° C./minute and it is finally subjected to a second temperature rise ranging from −20° C. to 100° C. at a heating rate of 5° C./minute. During the second temperature increase, the variation of the difference in power absorbed by the empty crucible and by the crucible containing the sample of wax is measured as a function of the temperature. The melting point of the compound is the temperature value corresponding to the top of the peak of the curve representing the variation in the difference in power absorbed as a function of the temperature.

For the purposes of the present invention, the term “hard wax” means a wax having, at 20° C., a hardness of greater than 5 MPa, especially ranging from 5 to 30 MPa, preferably greater than 6 MPa and better still ranging from 6 to 25 MPa.

The hardness of the wax is determined by measuring the compressive force, measured at 20° C. using the texturometer sold under the name TA-XT2 by the company Rheo, equipped with a stainless-steel cylinder 2 mm in diameter, traveling at a measuring speed of 0.1 mm/second, and penetrating the wax to a penetration depth of 0.3 mm.

The measuring protocol is as follows: the wax is melted at a temperature equal to the melting point of the wax +10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20⁰C before measuring the hardness or the tack.

The texturometer spindle is displaced at a speed of 0.1 mm/s, then penetrates the wax to a penetration depth of 0.3 mm. When the spindle has penetrated the wax to a depth of 0.3 mm, the spindle is held still for 1 second (corresponding to the relaxation time) and is then withdrawn at a speed of 0.5 mm/s.

The hardness value is the maximum compression force measured divided by the area of the texturometer cylinder in contact with the wax.

Waxes that may advantageously be used include waxes of plant origin such as beeswax, especially the product sold under the name White Beeswax SP 453P by the company Strahl & Pitsch or Cerabeil LOR by the company Baerlocher, black wheat wax such as the product sold by the company Codif, carnauba wax, candelilla wax, especially the commercial reference Candelilla Wax SP 75 G by the company Strahl & Pitsch, hydrogenated jojoba wax, sumach wax, the waxes obtained by hydrogenation of olive oil esterified with fatty alcohols bearing a C12 to C18 chain, sold by the company Sophim in the Phytowax range (12L44, 14L48, 16L55 and 18L57), rice bran wax, cetyl, stearyl and behenyl alcohols, laurel wax or ouricury wax.

Use may also be made of at least one ester of behenic acid and of glycerol, and in particular a mixture of esters of behenic acid and of glycerol, for instance the glyceryl dibehenate, tribehenin, glyceryl behenate mixture sold by the company Gattefossé under the reference Compritol 888 CG ATO.

When they are present, the waxes may be present in a content ranging from 0.01% to 40% by weight, preferably from 0.05% to 10% by weight and better still from 0.1% to 5% by weight relative to the total weight of the composition.

The fatty phase of the composition in accordance with the invention comprises at least one oil chosen from hydrocarbon-based oils of plant origin, preferably triglycerides, Guerbet alcohols, esters of a C8-C30 fatty acid and of a Guerbet alcohol, fatty alcohol ethers comprising from 6 to 20 carbon atoms, and linear C7-C17 alkanes, preferably chosen from hydrocarbon-based oils of plant origin, preferably triglycerides, Guerbet alcohols, esters of a C8-C30 fatty acid and of a Guerbet alcohol, and linear C7-C17 alkanes.

According to a particular embodiment of the invention, the composition comprises from 40% to 85% by weight of at least one oil chosen from hydrocarbon-based oils of plant origin, preferably triglycerides, Guerbet alcohols, esters of a C8-C30 fatty acid and of a Guerbet alcohol, fatty alcohol ethers comprising from 6 to 20 carbon atoms, and linear C7-C17 alkanes, preferably chosen from hydrocarbon-based oils of plant origin, preferably triglycerides, Guerbet alcohols, esters of a C8-C30 fatty acid and of a Guerbet alcohol, and linear C7-C17 alkanes.

According to a first specific embodiment, the composition comprises from 40% to 85% by weight and preferably from 45% to 75% by weight, relative to the total weight of the composition, of at least one oil chosen from hydrocarbon-based oils of plant origin, preferably triglycerides, Guerbet alcohols, and esters of a C8-C30 fatty acid and of a Guerbet alcohol.

According to a second specific embodiment, the composition comprises from 40% to 85% by weight and preferably from 45% to 75% by weight, relative to the total weight of the composition, of at least one oil chosen from hydrocarbon-based oils of plant origin, preferably triglycerides, Guerbet alcohols and esters of a C8-C30 fatty acid and of a Guerbet alcohol, and from 5% to 25% by weight and preferably from 5% to 10% by weight, relative to the total weight of the composition, of at least one oil chosen from linear C7-C17 alkanes.

According to a first specific embodiment of the invention, the anhydrous composition comprises:
from 3% to 15% by weight of at least one lipophilic gelling agent chosen from fatty acid esters of dextrin, preferably dextrin palmitate;
from 20% to 50% by weight and preferably from 30% to 40% by weight of at least one filler, preferably a single filler, chosen from porous spherical silica particles; and
from 40% to 85% by weight of at least one fatty phase containing at least 25% by weight of at least one oil chosen from hydrocarbon-based oils of plant origin, preferably triglycerides, Guerbet alcohols, esters of a C8-C30 fatty acid and of a Guerbet alcohol, fatty alcohol ethers comprising from 6 to 20 carbon atoms, and linear C7-C17 alkanes;
   the weight amounts being given relative to the total weight of the composition.

According to a particular embodiment, the anhydrous composition comprises:
from 3% to 15% by weight of at least one lipophilic gelling agent chosen from fatty acid esters of dextrin, preferably dextrin palmitate;
from 20% to 50% by weight and preferably from 30% to 40% by weight of at least one filler, preferably a single filler, chosen from porous spherical silica particles; and
from 40% to 85% by weight of at least one fatty phase comprising at least 25% by weight of at least one oil chosen from Guerbet alcohols and esters of a C8-C30 fatty acid and of a Guerbet alcohol;
   the weight amounts being given relative to the total weight of the composition.

Preferably, the anhydrous composition comprises:
from 3% to 15% by weight of at least one lipophilic gelling agent chosen from fatty acid esters of dextrin, preferably dextrin palmitate;
from 30% to 40% by weight of at least one filler, preferably a single filler, chosen from porous spherical silica particles; and
from 40% to 85% by weight of at least one fatty phase comprising at least 30% by weight of at least one oil chosen from Guerbet alcohols and esters of a C8-C30 fatty acid and of a Guerbet alcohol;
   the weight amounts being given relative to the total weight of the composition.

According to a particular embodiment, the anhydrous composition comprises:
from 3% to 15% by weight of at least one lipophilic gelling agent chosen from fatty acid esters of dextrin, preferably dextrin palmitate;
from 20% to 50% by weight and preferably from 30% to 40% by weight of at least one filler, preferably a single filler, chosen from porous spherical silica particles; and
from 40% to 85% by weight of at least one fatty phase containing at least 25% by weight of at least one oil chosen from hydrocarbon-based oils of plant origin, preferably triglycerides;
   the weight amounts being given relative to the total weight of the composition.

Preferably, the anhydrous composition comprises:
from 3% to 15% by weight of at least one lipophilic gelling agent chosen from fatty acid esters of dextrin, preferably dextrin palmitate;
from 30% to 40% by weight of at least one filler, preferably a single filler, chosen from porous spherical silica particles; and
from 40% to 85% by weight of at least one fatty phase containing at least 30% by weight of at least one oil chosen from hydrocarbon-based oils of plant origin, preferably triglycerides;
   the weight amounts being given relative to the total weight of the composition.

According to a particular embodiment, the anhydrous composition comprises:
from 3% to 15% by weight of at least one lipophilic gelling agent chosen from fatty acid esters of dextrin, preferably dextrin palmitate;
from 20% to 50% by weight and preferably from 30% to 40% by weight of at least one filler chosen from porous spherical silica particles; and
from 40% to 85% by weight of at least one fatty phase containing at least 25% by weight of at least two oils, one of which is chosen from hydrocarbon-based oils of plant origin, preferably triglycerides, and the other is chosen from linear C7-C17 alkanes;
   the weight amounts being given relative to the total weight of the composition.

Preferably, the anhydrous composition comprises:
from 3% to 15% by weight of at least one lipophilic gelling agent chosen from fatty acid esters of dextrin, preferably dextrin palmitate;
from 30% to 40% by weight of at least one filler chosen from porous spherical silica particles; and
from 40% to 85% by weight of at least one fatty phase containing at least 25% by weight of at least one oil chosen from hydrocarbon-based oils of plant origin, preferably triglycerides, and at least 5% by weight of at least one oil chosen from linear C7-C17 alkanes;
   the weight amounts being given relative to the total weight of the composition.

According to a second specific embodiment of the invention, the anhydrous composition comprises:
from 3% to 15% by weight of at least one lipophilic gelling agent chosen from triesters of a C8-C30 fatty acid and of mono- or polyglycerol, such as glyceryl trihydroxystearate;
from 20% to 50% by weight and preferably from 30% to 40% by weight of at least one filler chosen from powders of an N-acylamino acid bearing a C8-C22 acyl group; and
from 40% to 85% by weight of at least one fatty phase containing at least 25% by weight of at least one oil chosen from hydrocarbon-based oils of plant origin, preferably triglycerides, Guerbet alcohols, esters of a C8-C30 fatty acid and of a Guerbet alcohol, fatty alcohol ethers comprising from 6 to 20 carbon atoms, and linear C7-C17 alkanes;
   the weight amounts being given relative to the total weight of the composition.

According to a particular embodiment, the anhydrous composition comprises:
from 3% to 15% by weight of at least one lipophilic gelling agent chosen from triesters of a C8-C30 fatty acid and of mono- or polyglycerol, such as glyceryl trihydroxystearate;
from 20% to 50% by weight and preferably from 30% to 40% by weight of at least one filler chosen from powders of an N-acylamino acid bearing a C8-C22 acyl group; and
from 40% to 85% by weight of at least one fatty phase containing at least 25% by weight of at least two oils, one of which is chosen from hydrocarbon-based oils of plant origin, preferably triglycerides, and the other is chosen from linear C7-C17 alkanes;
   the weight amounts being given relative to the total weight of the composition.

Preferably, the anhydrous composition comprises:
from 3% to 15% by weight of at least one lipophilic gelling agent chosen from triesters of a C8-C30 fatty acid and of mono- or polyglycerol, such as glyceryl trihydroxystearate;
from 30% to 40% by weight of at least one filler chosen from powders of an N-acylamino acid bearing a C8-C22 acyl group; and
from 45% to 85% by weight of at least one fatty phase containing at least 40% by weight of at least one oil chosen from hydrocarbon-based oils of plant origin, preferably triglycerides, and at least 5% by weight of at least one oil chosen from linear C7-C17 alkanes;
   the weight amounts being given relative to the total weight of the composition.

According to a particular embodiment, the anhydrous composition comprises:
from 3% to 15% by weight of at least one lipophilic gelling agent chosen from triesters of a fatty acid and of mono- or polyglycerol, such as glyceryl trihydroxystearate;
from 20% to 50% by weight and preferably from 30% to 40% by weight of at least one filler chosen from powders of an N-acylamino acid bearing a C8-C22 acyl group; and
from 40% to 85% by weight of at least one fatty phase containing at least 25% by weight of at least one oil chosen from fatty alcohol ethers comprising from 6 to 20 carbon atoms;
   the weight amounts being given relative to the total weight of the composition.

Preferably, the anhydrous composition comprises:
from 3% to 15% by weight of at least one lipophilic gelling agent chosen from triesters of a fatty acid and of mono- or polyglycerol, such as glyceryl trihydroxystearate;
from 30% to 40% by weight of at least one filler chosen from powders of an N-acylamino acid bearing a C8-C22 acyl group; and
from 40% to 85% by weight of at least one fatty phase containing at least 25% by weight of at least one oil chosen from fatty alcohol ethers comprising from 6 to 20 carbon atoms;
   the weight amounts being given relative to the total weight of the composition.

According to a third specific embodiment of the invention, the anhydrous composition comprises:
from 3% to 15% by weight of at least one lipophilic gelling agent chosen from fatty acid esters of dextrin, preferably dextrin myristate;
from 20% to 50% by weight and preferably from 30% to 40% by weight of at least one filler chosen from powders of an N-acylamino acid bearing a C8-C22 acyl group; and
from 40% to 85% by weight of at least one fatty phase containing at least 25% by weight of at least one oil chosen from hydrocarbon-based oils of plant origin, preferably triglycerides, Guerbet alcohols, esters of a C8-C30 fatty acid and of a Guerbet alcohol, fatty alcohol ethers comprising from 6 to 20 carbon atoms, and linear C7-C17 alkanes;
   the weight amounts being given relative to the total weight of the composition.

According to a particular embodiment, the anhydrous composition comprises:
from 3% to 15% by weight of at least one lipophilic gelling agent chosen from fatty acid esters of dextrin, preferably dextrin myristate;
from 20% to 50% by weight and preferably from 30% to 40% by weight of at least one filler chosen from powders of an N-acylamino acid bearing a C8-C22 acyl group; and
from 40% to 85% by weight of at least one fatty phase comprising at least 25% by weight of at least one oil chosen from Guerbet alcohols and esters of a C8-C30 fatty acid and of a Guerbet alcohol;
   the weight amounts being given relative to the total weight of the composition.

Preferably, the anhydrous composition comprises:
from 3% to 15% by weight of at least one lipophilic gelling agent chosen from fatty acid esters of dextrin, preferably dextrin myristate;
from 30% to 40% by weight of at least one filler chosen from powders of an N-acylamino acid bearing a C8-C22 acyl group; and
from 50% to 85% by weight of at least one fatty phase comprising at least 50% by weight of at least one oil chosen from Guerbet alcohols and esters of a C8-C30 fatty acid and of a Guerbet alcohol;
   the weight amounts being given relative to the total weight of the composition.

According to a particular embodiment, the anhydrous composition comprises:
from 3% to 15% by weight of at least one lipophilic gelling agent chosen from fatty acid esters of dextrin, preferably dextrin myristate;
from 20% to 50% by weight and preferably from 20% to 30% by weight of at least one filler chosen from powders of an N-acylamino acid bearing a C8-C22 acyl group; and
from 40% to 85% by weight of at least one fatty phase containing at least 25% by weight of at least one oil chosen from hydrocarbon-based oils of plant origin, preferably triglycerides;
   the weight amounts being given relative to the total weight of the composition.

Preferably, the anhydrous composition comprises:
from 3% to 15% by weight of at least one lipophilic gelling agent chosen from fatty acid esters of dextrin, preferably dextrin myristate;
from 20% to 30% by weight of at least one filler chosen from powders of an N-acylamino acid bearing a C8-C22 acyl group; and
from 60% to 85% by weight of at least one fatty phase containing at least 60% by weight of at least one oil chosen from hydrocarbon-based oils of plant origin, preferably triglycerides;
   the weight amounts being given relative to the total weight of the composition.

According to a fourth specific embodiment of the invention, the anhydrous composition comprises:
from 3% to 15% by weight of at least one lipophilic gelling agent chosen from triesters of a fatty acid and of mono- or polyglycerol, such as glyceryl trihydroxystearate;
from 20% to 50% by weight of at least one filler chosen from powders of an N-acylamino acid bearing a C8-C22 acyl group; and
from 40% to 85% by weight of at least one fatty phase containing at least 20% by weight of at least one oil chosen from hydrocarbon-based oils of plant origin, preferably triglycerides, Guerbet alcohols, esters of a C8-C30 fatty acid and of a Guerbet alcohol, fatty alcohol ethers comprising from 6 to 20 carbon atoms, and linear C7-C17 alkanes;
   the weight amounts being given relative to the total weight of the composition.

According to a particular embodiment, the anhydrous composition comprises:
from 3% to 15% by weight of at least one lipophilic gelling agent chosen from triesters of a fatty acid and of mono- or polyglycerol, such as glyceryl trihydroxystearate;
from 20% to 50% by weight and preferably from 20% to 30% by weight of at least one filler chosen from powders of an N-acylamino acid bearing a C8-C22 acyl group; and
from 40% to 85% by weight of at least one fatty phase containing at least 20% by weight of at least one oil chosen from linear C7-C17 alkanes;
   the weight amounts being given relative to the total weight of the composition.

Preferably, the anhydrous composition comprises:
from 3% to 15% by weight of at least one lipophilic gelling agent chosen from triesters of a fatty acid and of mono- or polyglycerol, such as glyceryl trihydroxystearate;
from 20% to 30% by weight of at least one filler chosen from powders of an N-acylamino acid bearing a C8-C22 acyl group; and
from 40% to 85% by weight of at least one fatty phase containing at least 20% by weight of at least one oil chosen from linear C7-C17 alkanes;
   the weight amounts being given relative to the total weight of the composition.

According to a fifth specific embodiment of the invention, the anhydrous composition comprises:
from 3% to 15% by weight of at least one lipophilic gelling agent chosen from fatty acid esters of dextrin, preferably dextrin myristate;
from 20% to 50% by weight and preferably from 40% to 50% by weight of at least one filler chosen from spherical cellulose particles; and
from 40% to 85% by weight of at least one fatty phase containing at least 25% by weight of at least one oil chosen from hydrocarbon-based oils of plant origin, preferably triglycerides, Guerbet alcohols, esters of a C8-C30 fatty acid and of a Guerbet alcohol, fatty alcohol ethers comprising from 6 to 20 carbon atoms, and linear C7-C17 alkanes;
   the weight amounts being given relative to the total weight of the composition.

According to a particular embodiment, the anhydrous composition comprises:
from 3% to 15% by weight of at least one lipophilic gelling agent chosen from fatty acid esters of dextrin, preferably dextrin myristate;
from 20% to 50% by weight and preferably from 40% to 50% by weight of at least one filler chosen from spherical cellulose particles; and
from 40% to 85% by weight of at least one fatty phase containing at least 25% by weight of at least one oil chosen from hydrocarbon-based oils of plant origin, preferably triglycerides;
   the weight amounts being given relative to the total weight of the composition.

Preferably, the anhydrous composition comprises:
from 3% to 15% by weight of at least one lipophilic gelling agent chosen from fatty acid esters of dextrin, preferably dextrin myristate;
from 40% to 50% by weight of at least one filler chosen from spherical cellulose particles; and
from 40% to 85% by weight of at least one fatty phase containing at least 40% by weight of at least one oil chosen from hydrocarbon-based oils of plant origin, preferably triglycerides;
   the weight amounts being given relative to the total weight of the composition.

According to a sixth specific embodiment of the invention, the anhydrous composition comprises:
from 3% to 15% by weight of at least one lipophilic gelling agent chosen from triesters of a fatty acid and of mono- or polyglycerol, such as glyceryl trihydroxystearate;
from 20% to 50% by weight and preferably from 30% to 40% by weight of at least one filler chosen from spherical cellulose particles; and
from 40% to 85% by weight of at least one fatty phase containing at least 25% by weight of at least one oil chosen from hydrocarbon-based oils of plant origin, preferably triglycerides, Guerbet alcohols, esters of a C8-C30 fatty acid and of a Guerbet alcohol, fatty alcohol ethers comprising from 6 to 20 carbon atoms, and linear C7-C17 alkanes;
   the weight amounts being given relative to the total weight of the composition.

According to a particular embodiment, the anhydrous composition comprises:
from 3% to 15% by weight of at least one lipophilic gelling agent chosen from triesters of a fatty acid and of mono- or polyglycerol, such as glyceryl trihydroxystearate;
from 20% to 50% by weight and preferably from 30% to 40% by weight of at least one filler chosen from spherical cellulose particles; and
from 40% to 85% by weight of at least one fatty phase containing at least 25% by weight of at least two oils, one of which is chosen from hydrocarbon-based oils of plant origin, preferably triglycerides, and the other is chosen from linear C7-C17 alkanes;
   the weight amounts being given relative to the total weight of the composition.

Preferably, the anhydrous composition comprises:
from 3% to 15% by weight of at least one lipophilic gelling agent chosen from triesters of a fatty acid and of mono- or polyglycerol, such as glyceryl trihydroxystearate;
from 30% to 40% by weight of at least one filler chosen from spherical cellulose particles; and
from 45% to 85% by weight of at least one fatty phase containing at least 25% by weight of at least one oil chosen from hydrocarbon-based oils of plant origin, preferably triglycerides, and at least 20% by weight of at least one oil chosen from linear C7-C17 alkanes;
   the weight amounts being given relative to the total weight of the composition.

According to a seventh specific embodiment of the invention, the anhydrous composition comprises:
from 3% to 15% by weight of at least one lipophilic gelling agent chosen from fatty acid esters of dextrin, preferably dextrin palmitate;
from 20% to 50% by weight and preferably from 30% to 40% by weight of at least one filler chosen from spherical cellulose particles; and
from 40% to 85% by weight of at least one fatty phase containing at least 25% by weight of at least one oil chosen from hydrocarbon-based oils of plant origin, preferably triglycerides, Guerbet alcohols, esters of a C8-C30 fatty acid and of a Guerbet alcohol, fatty alcohol ethers comprising from 6 to 20 carbon atoms, and linear C7-C17 alkanes;
   the weight amounts being given relative to the total weight of the composition.

According to a particular embodiment, the anhydrous composition comprises:
from 3% to 15% by weight of at least one lipophilic gelling agent chosen from fatty acid esters of dextrin, preferably dextrin palmitate;
from 20% to 50% by weight and preferably from 30% to 40% by weight of at least one filler chosen from spherical cellulose particles; and
from 40% to 85% by weight of at least one fatty phase comprising at least 25% by weight of at least one oil chosen from Guerbet alcohols and esters of a C8-C30 fatty acid and of a Guerbet alcohol;
   the weight amounts being given relative to the total weight of the composition.

Preferably, the anhydrous composition comprises:
from 3% to 15% by weight of at least one lipophilic gelling agent chosen from fatty acid esters of dextrin, preferably dextrin palmitate;
from 30% to 40% by weight of at least one filler chosen from spherical cellulose particles; and
from 40% to 85% by weight of at least one fatty phase comprising at least 30% by weight of at least one oil chosen from Guerbet alcohols and esters of a C8-C30 fatty acid and of a Guerbet alcohol;
   the weight amounts being given relative to the total weight of the composition.

The composition according to the invention may also contain one or more polyols comprising from 2 to 8 carbon atoms. The term “polyol” should be understood as meaning any organic molecule comprising at least two free hydroxyl groups. Examples of polyols that may be mentioned include glycerol, glycols, for instance butylene glycol, propylene glycol, isoprene glycol, dipropylene glycol, hexylene glycol, polyethylene glycols and polypropylene glycol and especially dipropylene glycol, 1,2-propanediol and 1,3-propanediol. According to a particular embodiment of the invention, the polyol is chosen from glycerol and 1,3-propanediol. Preferably, the polyol is glycerol.

Examples that may be mentioned include the glycerol sold under the name Glycerine 4810 by the company Oleon or under the name Palmera G995V by the company KLK Oleo or alternatively under the name Refined Glycerine 99.5% PH. EURO by the company Cargill.

Mention may also be made of the 1,3-propanediol sold under the name Zemea Propanediol by the company Dupont Tate and Lyle Bio Products.

Mention may also be made of the butylene glycol sold under the name 1,3-Butylene Glycol by the company Alzo or alternatively Daicel.

Mention may also be made of the propylene glycol sold under the name Radianol 4710 by the company Oleon.

The amount of polyols may range, for example, from 0% to 20% by weight, especially from 0.1% to 20% by weight, preferably from 3% to 17% by weight, better still from 4% to 15% by weight and even better still from 5% to 10% by weight, relative to the total weight of the composition.

The composition in accordance with the invention may also comprise one or more primary alcohols, i.e. an alcohol comprising from 1 to 6 carbon atoms, such as ethanol, propanol, isopropanol, butanol, pentanol or hexanol, and in particular ethanol and isopropanol. It is preferably ethanol. The addition of such an alcohol may especially be suitable when the composition according to the invention is used as a product for the body, the face or the hair.

The amount of primary alcohols may range, for example, from 0% to 35% by weight, especially from 0.1% to 35% by weight, preferably from 1% to 15% by weight and even more preferentially from 5% to 10% by weight, relative to the total weight of the composition.

The composition of the invention may contain one or more adjuvants that are common in the cosmetic and dermatological fields, such as surfactants; moisturizers; hydrophilic or lipophilic active agents; free-radical scavengers; antioxidants; preserving agents; fragrances; film-forming agents; nacres; pigments; dyes; and mixtures thereof.

The composition in accordance with the invention may comprise one or more active agents.

Nonlimiting examples of active agents that may be mentioned include ascorbic acid and derivatives thereof such as 5,6-di-O-dimethylsilyl ascorbate (sold especially by the company Exsymol under the reference PRO-AA), the potassium salt of dl-alpha-tocopheryl-21-ascorbyl phosphate (sold especially by the company Senju Pharmaceutical under the reference Sepivital EPC), magnesium ascorbyl phosphate, sodium ascorbyl phosphate (sold especially by the company Roche under the reference Stay-C 50); phloroglucinol; enzymes; and mixtures thereof. According to one preferred embodiment of the invention, use is made, among oxidation-sensitive hydrophilic active agents, of ascorbic acid. The ascorbic acid can be of any nature. Thus, it may be of natural origin in powder form or in the form of orange juice, preferably orange juice concentrate. It may also be of synthetic origin, preferably in powder form.

As other active agents that may be used in the composition of the invention, examples that may be mentioned include moisturizers, such as protein hydrolyzates; natural extracts; anti-inflammatories; oligomeric proanthocyanidins; vitamins such as vitamin A (retinol), vitamin E (tocopherol) and other antioxidants such as extract of rosemary, vitamin B5 (panthenol), vitamin B3 (niacinamide), derivatives of these vitamins (especially esters) and mixtures thereof; urea, caffeine, adenosine, depigmenting agents such as kojic acid, hydroquinone and caffeic acid; salicylic acid and derivatives thereof; alpha-hydroxy acids, such as lactic acid and glycolic acid and derivatives thereof; retinoids, such as carotenoids and vitamin A derivatives; hydrocortisone; melatonin; extracts of algae, of fungi, of plants, of yeasts, of bacteria; steroids; antibacterial active agents; matt-effect agents, for instance fibers; tensioning agents, waxes; UV-screening agents, essential oils; ceramides; and mixtures thereof.

The amounts of these various adjuvants and/or active agents are those conventionally used in the fields under consideration. In particular, these amounts vary according to the desired aim and may range, for example, from 0.01% to 20% and preferably from 0.1% to 10% by weight relative to the total weight of the composition.

Needless to say, a person skilled in the art will take care to select the optional adjuvant(s) and/or active agent(s) added to the composition according to the invention such that the advantageous properties intrinsically associated with the composition in accordance with the invention are not, or are not substantially, adversely affected by the envisioned addition.

The composition according to the invention may be used for any topical application; in particular, it may constitute a cosmetic or dermatological composition, preferably a cosmetic composition, and in particular in the cosmetic field with immediate perceived efficacy. It may in particular be used for caring for and/or removing makeup from the skin, the lips and/or the eyes, and also as a haircare composition. It may also be used as a deodorant or as an antisun product, and also for cleansing the skin.

The composition in accordance with the invention may especially be used as a product for cosmetic use for caring for the skin with regard to antiaging care, greasy skin, antisun protection, antiperspirants and deodorants, hair and/or scalp products, and also styling products, fragrancing products and makeup products.

A subject of the invention is also the cosmetic use of a composition as defined above, for caring for, removing makeup from and/or cleansing the skin, the lips and/or the eyes, and/or for haircare.

A subject of the present invention is also a cosmetic process for removing makeup from and/or caring for the skin, the lips and/or the eyes, in which a composition as defined above is applied to the skin, the lips and/or the eyes.

A subject of the present invention is also a cosmetic haircare process, in which a composition as defined above is applied to the hair.

A subject of the present invention is also a cosmetic treatment process for hiding skin color imperfections and/or skin relief imperfections.

According to a preferred embodiment of the invention, the composition is a composition for caring for bodily and/or facial skin, preferably facial skin.

A subject of the present invention is also an aqueous composition in the form of a dispersion of at least one anhydrous composition as defined previously in an aqueous phase.

The composition in accordance with the invention may be obtained in the usual manner for those skilled in the art.

According to a particular embodiment, the composition in accordance with the invention is obtained according to the following process:

1) heating the fatty phase until a temperature of 70° C. to 80° C. is obtained, depending on the case;
2) adding the gelling agent and gelling at 70° C. to 80° C., depending on the case, for 15 minutes with an emulsifying machine, the maximum speed being adapted to the volume manufactured;
3) cooling until a temperature of 35° C. is obtained;
4) adding the fillers at 35° C. Dispersing until the lumps have disappeared and a homogeneous fluid of smooth, matt macroscopic appearance is obtained;
5) emptying the tank.

The examples that follow will allow the invention to be understood more clearly, without, however, being limiting in nature. The raw materials are referred to by their chemical or INCI name. The amounts indicated are weight percentages of raw materials, unless otherwise mentioned.

### EXAMPLES

### Examples 1 to 4

Compositions 1 to 4 below were prepared.

| Composition | 1 | 2 | 3 | 4 |
|---|---|---|---|---|
| Silica | 35 | 35 | 35 | 35 |
| (Silica Beads SB-300 from the | | | | |
| company Miyoshi Kasei) | | | | |
| Dextrin palmitate | 7.5 | 7.5 | 7 | 7 |
| (Rheopearl TL2-OR from Chiba | | | | |
| Flour Milling) | | | | |
| Caprylic/capric triglyceride | — | 28.2 | 33.45 | 28.60 |
| (Myritol 318 from Cognis (BASF)) | | | | |
| Octyldodecyl myristate | 33.45 | — | — | — |
| (MOD from Gattefossé) | | | | |
| Prunus armeniaca kernel oil | 10 | 10 | — | — |
| (Lipovol P from Lipo Chemicals) | | | | |
| Isopropyl palmitate | 14 | 14.25 | 14 | 14.35 |
| (Isopropyl palmitate from Cognis) | | | | |
| Undecane (and) tridecane | — | 5 | — | 5 |
| (Cetiol UT from Cognis) | | | | |
| Antioxidant | 0.05 | 0.05 | 0.05 | 0.05 |

### Preparation Process

The fatty phase is homogenized. After introducing the gelling agent, gelling phase once the desired temperature has been reached (70° C. to 80° C., depending on the case). Cooling phase, followed by adding any other ingredient of the formulation and then the fillers (temperature ranging from 50° C. to 32° C.).

### Characterization of the Optical Properties

The optical properties of compositions 1 to 4 were characterized by means of the Haze measurement (veil effect) with a commercial Hazemeter machine.

The measurements were taken according to the following protocol: on a transparent plastic film (Byk), a coat with a wet thickness of 25.4 μm of the composition whose haze it is desired to evaluate is spread out, using an automatic spreader. The coat is left to dry for 1 hour at room temperature, and measurement of the haze index is then taken using a Byk Gardner brand Haze-gard.

The values obtained for compositions 1 to 10 are the following:

| Composition | 1 | 2 | 3 | 4 |
|---|---|---|---|---|
| Measured Haze | 60 | 80 | 65.2 | 89.3 |

As a guide, it is considered that between 20 and 60, a weak soft-focus effect was obtained, between 60 and 80, a good soft-focus effect was obtained, and at and above 90, a very good soft-focus effect was obtained.

These measurements thus show that the compositions in accordance with the invention make it possible to obtain a large soft-focus effect.

### Sensory Evaluation:

The sensory properties of compositions 1 to 4 were evaluated after applying them to the skin, and are summarized in the table below:

| Composition | Sensory evaluation |
|---|---|
| 1 | rapid penetration, leaves a considerable soft film with |
| | a powdery, velvety, non-tacky feel |
| 2 | rapid penetration, very soft skin finish and very |
| | powdery, non-tacky. |
| 3 | rapid penetration, non-tacky, non-glossy, soft, powdery |
| | skin finish effect. |
| 4 | rapid penetration, very soft skin finish and very |
| | powdery, non-tacky. |

The cosmetic properties of compositions 1 to 4 are good, especially in terms of speed of penetration, absence of tack, and softness.

### Stability

The stability of compositions 1 to 4 was evaluated over a period of 2 months at room temperature (25° C.). All the compositions are stable after 2 months of storage at 25° C.

### Examples 5 to 9

Compositions 5 to 9 below were prepared.

| Composition | 5 | 6 | 7 |
|---|---|---|---|
| Lauroyl lysine | 35 | 35 | 35 |
| (Amihope LL from Ajinomoto) | | | |
| Dextrin myristate | — | 7 | — |
| (Rheopearl MKL2 from Chiba Flour Milling) | | | |
| Trihydroxystearin | 6.5 | — | 7.8 |
| (Thixcin R from Elementis) | | | |
| Caprylic/capric triglyceride | — | — | 48.2 |
| (Myritol 318 from Cognis (BASF)) | | | |
| Dicaprylyl Ether | 30 | — | — |
| (Cetiol OE from Cognis) | | | |
| Octyldodecyl myristate | — | 58 | — |
| (MOD from Gattefossé) | | | |
| Isopropyl palmitate | 28.5 | — | — |
| (Isopropyl palmitate from Cognis) | | | |
| Undecane (and) tridecane | — | — | 9 |
| (Cetiol UT from Cognis) | | | |

| Composition | 8 | 9 |
|---|---|---|
| Lauroyl lysine | 25 | 20 |
| (Amihope LL from Ajinomoto) | | |
| Dextrin myristate | 7.5 | — |
| (Rheopearl MKL2 from Chiba Flour Milling) | | |
| Trihydroxystearin | — | 9.6 |
| (Thixcin R from Elementis) | | |
| Caprylic/capric triglyceride | 67.5 | — |
| (Myritol 318 from Cognis (BASF)) | | |
| Dicaprylyl Ether | — | — |
| (Cetiol OE from Cognis) | | |
| Octyldodecyl myristate | — | — |
| (MOD from Gattefossé) | | |
| Isopropyl palmitate | — | — |
| (Isopropyl palmitate from Cognis) | | |
| Isopropyl isostearate | — | 50.4 |
| (Radia 7739 from Oleon) | | |
| Undecane (and) tridecane | — | 20 |
| (Cetiol UT from Cognis) | | |

### Preparation Process

The fatty phase is homogenized. After introducing the gelling agent, gelling phase once the desired temperature has been reached (70° C. to 80° C., depending on the case).

Cooling phase, followed by adding any other ingredient of the formulation and then the fillers (temperature ranging from 50° C. to 32° C.).

### Characterization of the Optical Properties

The optical properties of compositions 5 to 9 were characterized by means of the Haze measurement (veil effect) with a commercial Hazemeter machine.

The measurements were taken according to the following protocol: on a transparent plastic film (Byk), a coat with a wet thickness of 25.4 μm of the composition whose haze it is desired to evaluate is spread out, using an automatic spreader. The coat is left to dry for 1 hour at room temperature, and measurement of the haze index is then taken using a Byk Gardner brand Haze-gard.

The values obtained for compositions 5 to 9 are the following:

| Composition | 5 | 6 | 7 | 8 | 9 |
|---|---|---|---|---|---|
| Measured Haze | 85.1 | 79.1 | 90.5 | 74.23 | 79.4 |

As a guide, it is considered that between 20 and 60, a weak soft-focus effect was obtained, between 60 and 80, a good soft-focus effect was obtained, and at and above 90, a very good soft-focus effect was obtained.

These measurements thus show that the compositions in accordance with the invention make it possible to obtain a large soft-focus effect.

### Sensory Evaluation:

The sensory properties of compositions 5 to 9 were evaluated after applying them to the skin, and are summarized in the table below:

| Composition | Sensory evaluation |
|---|---|
| 5 | Leaves a soft film, powdery, velvety, non-tacky, |
| | non-glossy, non-greasy feel. |
| 6 | Rapid penetration with a film having a slightly |
| | tacky skin finish. |
| 7 | Leaves a soft film, powdery, velvety, non-tacky, |
| | non-glossy, non-greasy feel. |
| 8 | Film-forming skin finish. |
| 9 | Very soft, film-forming, non-glossy, slightly |
| | tacky skin finish. |

### Stability

The stability of compositions 5 to 9 was evaluated over a period of 2 months at room temperature (25° C.). All the compositions are stable after 2 months of storage at 25° C.

### Examples 10 and 11

Compositions 10 and 11 below were prepared.

| Composition | 10 | 11 |
|---|---|---|
| Cellulose | 50 | — |
| (Cellulobeads D-10 from Daito Kasei Kogyo) | | |
| Cellulose | — | 35 |
| (Cellulobeads D-5 from Daito Kasei) | | |
| Caprylic/capric triglyceride | 45 | — |
| (Myritol 318 from Cognis (BASF)) | | |
| Octyldodecyl myristate | — | 33.45 |
| (MOD from Gattefossé) | | |
| Isopropyl palmitate | — | 14 |
| (Isopropyl palmitate from Cognis) | | |
| Prunus armeniaca kernel oil | — | 10 |
| (Lipovol P from Lipo Chemicals) | | |
| Undecane (and) tridecane | — | — |
| (Cetiol UT from Cognis) | | |
| Trihydroxystearin | — | — |
| (Thixcin R from Elementis) | | |
| Dextrin palmitate | — | 7.5 |
| (Rheopearl TL2-OR from Chiba Flour Milling) | | |
| Dextrin myristate | 5 | — |
| (Rheopearl MKL2 from Chiba Flour Milling) | | |

### Preparation Process

The fatty phase is homogenized. After introducing the gelling agent, gelling phase once the desired temperature has been reached (70° C. to 80° C., depending on the case).

Cooling phase, followed by adding any other ingredient of the formulation and then the fillers (temperature ranging from 50° C. to 32° C.).

Characterization of the optical properties

The optical properties of compositions 10 and 11 were characterized by means of the Haze measurement (veil effect) with a commercial Hazemeter machine.

The measurements were taken according to the following protocol: on a transparent plastic film (Byk), a coat with a wet thickness of 25.4 μm of the composition whose haze it is desired to evaluate is spread out, using an automatic spreader. The coat is left to dry for 1 hour at room temperature, and measurement of the haze index is then taken using a Byk Gardner brand Haze-gard.

The values obtained for compositions 10 and 11 are the following:

| Composition | 10 | 11 |
|---|---|---|
| Measured Haze | 85.2 | 77 |

As a guide, it is considered that between 20 and 60, a weak soft-focus effect was obtained, between 60 and 80, a good soft-focus effect was obtained, and at and above 90, a very good soft-focus effect was obtained.

These measurements thus show that the compositions in accordance with the invention make it possible to obtain a large soft-focus effect.

### Sensory Evaluation:

The sensory properties of compositions 10 and 11 were evaluated after applying them to the skin, and are summarized in the table below:

| Composition | Sensory evaluation |
|---|---|
| 10 | Very soft, film-forming, non-glossy skin finish. |
| 11 | Glidant powdery skin finish. |

### Stability

The stability of compositions 10 and 11 was evaluated over a period of 2 months at room temperature (25° C.). All the compositions are stable after 2 months of storage at 25° C.

1. An anhydrous composition, comprising: based on a total weight of the composition,
from 3% to 15% by weight of at least one lipophilic gelling agent;
from 20% to 50% by weight of at least one filler selected from the group consisting of spherical porous silica particles, spherical cellulose particles, and powders of an N-acylamino acid comprising a C8-C22 acyl group; and
from 40% to 85% by weight of at least one fatty phase containing at least 20% by weight of at least one oil selected from the group consisting of a hydrocarbon-based oil of plant origin, a Guerbet alcohol, an ester of a C8-C30 fatty acid and of a Guerbet alcohol, a fatty alcohol ether comprising from 6 to 20 carbon atoms, and a linear C7-C17 alkane.

2. The composition of claim 1, wherein the at least one lipophilic gelling agent are selected from the group consisting of a fatty acid ester of dextrin and a fatty acid of a trimester of a C8-C30 fatty acid and of mono- or polyglycerol with 2 to 10 glycerol units.

3. The composition of claim 1, wherein the at least one lipophilic gelling agent is a fatty acid ester of dextrin, which is a mono- or polyester of dextrin and of at least one fatty acid to formula (C): 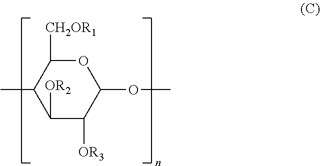
wherein
n is an integer ranging from 3 to 150;
R1, R2 and R3 each independently represent a hydrogen atom or an acyl group (R—CO—) in which R is a linear or branched, saturated or unsaturated hydrocarbon-based group containing from 6 to 50 carbon atoms, with the proviso that at least one of R1, R2 or R3 is not a hydrogen atom.

4. The composition of claim 3, wherein the fatty acid ester of dextrin is a compound of formula (C) in which:
n ranges from 25 to 35;
the acyl group R—CO— is selected from the group consisting of caprylyl, caproyl, lauroyl, myristyl, palmityl, stearyl, eicosanyl, docosanoyl, isovaleryl, 2-ethylbutyryl, ethylmethylacetyl, isoheptanyl, 2-ethylhexanyl, isononanyl, isodecanyl, isotridecanyl, isomyristyl, isopalmityl, isostearyl, isohexanyl, decenyl, dodecenyl, tetradecenyl, myristyl, hexadecenoyl, palmitolyl, oleyl, elaidyl, eicosenyl, sorbyl, linoleyl, linolenyl, punicyl, arachidonyl and stearolyl, and a mixture thereof.

5. The composition of claim 2, wherein the at least one lipophilic gelling agent is a fatty acid of a triester of a C8-C30 fatty acid and of mono- or polyglycerol with 2 to 10 glycerol units, which is a linear or branched, saturated or unsaturated acid, comprising from 10 to 24 carbon atoms.

6. The composition of claim 5, wherein the trimester of a C8-C30 fatty acid and of mono- or polyglycerol is a trimester of a fatty acid and of monoglycerol.

7. The composition of claim 1, wherein the filler is spherical porous silica particles.

8. The composition of claim 1, wherein the filler is spherical cellulose particles.

9. The composition of claim 1, wherein the filler is powders of an N-acylamino acid comprising a C8-C22 acyl group.

10. The composition of claim 1, comprising from 45% to 85% by weight of the at least one fatty phase.

11. The composition of claim 1, wherein the fatty phase comprises at least 20% by weight of a hydrocarbon-based oil of plant origin, a Guerbet alcohol, an ester of a C8-C30 fatty acid and of a Guerbet alcohol, and a linear C7-C17 alkane.

12. The composition of claim 1, wherein the fatty phase comprises at least 25% by weight of at least two oils selected from the group consisting of a hydrocarbon-based oil of plant origin, a Guerbet alcohol, an ester of a C8-C30 fatty acid and of a Guerbet alcohol, and a linear C7-C17 alkane.

13. The composition of claim 1, wherein the fatty phase comprises a hydrocarbon-based oil of plant origin, which is selected from the group consisting of squalane, a liquid triglyceride of a fatty acid comprising from 4 to 30 carbon atoms, jojoba oil, babassu oil, sunflower oil, olive oil, coconut oil, Brazil nut oil, marula oil, corn oil, soybean oil, marrow oil, grapeseed oil, linseed oil, sesame seed oil, hazelnut oil, apricot oil, macadamia nut oil, arara oil, coriander oil, castor oil, avocado oil, a caprylic/capric acid triglyceride, and shea butter oil.

14. The composition of claim 1, wherein the fatty phase comprises a linear C7-C17 alkane, which comprises from 9 to 15 carbon atoms.

15. The composition of claim 1, wherein the fatty phase comprises a Guerbet alcohol and an ester of a C8-C30 fatty acid and of a Guerbet alcohol, which are octyldodecanol and an octyldodecanol ester, respectively.

16. The composition of claim 1, wherein the fatty phase comprises a fatty alcohol ether, which is an ether of a fatty alcohol comprising from 8 to 12.

17. The composition of claim 1, comprising:
from 3% to 15% by weight of the at least one lipophilic gelling agent, which is a fatty acid ester of dextrin;
from 30% to 40% by weight of the at least one filler, which is porous spherical silica particles; and
from 40% to 85% by weight of the at least one fatty phase containing at least 25% by weight of at least one hydrocarbon-based oil of plant origin, and at least 5% by weight of at least one linear C7-C17 alkane.

18. The composition of claim 1, comprising:
from 3% to 15% by weight of the at least one lipophilic gelling agent, which is a triester of a C8-C30 fatty acid and of mono- or polyglycerol;
from 30% to 40% by weight of the at least one filler, which is powders of an N-acylamino acid comprising a C8-C22 acyl group; and
from 45% to 85% by weight of the at least one fatty phase containing at least 40% by weight of at least one oil chosen from hydrocarbon-based oil of plant origin, and at least 5% by weight of at least one linear C7-C17 alkane.

19. The composition of claim 1, comprising:
from 3% to 15% by weight of the at least one lipophilic gelling agent, which is a fatty acid ester of dextrin;
from 40% to 50% by weight of the at least one filler, which is spherical cellulose particles; and
from 40% to 85% by weight of the at least one fatty phase containing at least 40% by weight of at least one hydrocarbon-based oil of plant origin.

20. The composition of claim 1, comprising:
from 3% to 15% by weight of the at least one lipophilic gelling agent, which is a triester of a C8-C30 fatty acid and of mono- or polyglycerol;
from 30% to 40% by weight of the at least one filler, which is spherical cellulose particles; and
from 45% to 85% by weight of the at least one fatty phase containing at least 25% by weight of at least one hydrocarbon-based oil of plant origin, and at least 20% by weight of at least one linear C7-C17 alkanes.

21. The composition of claim 1, comprising:
from 3% to 15% by weight of the at least one lipophilic gelling agent, which is a triester of a C8-C30 fatty acid and of mono- or polyglycerol;
from 30% to 40% by weight of the at least one filler, which is powders of an N-acylamino acid comprising a C8-C22 acyl group; and
from 40% to 85% by weight of the at least one fatty phase containing at least 25% by weight of at least one fatty alcohol ether comprising from 6 to 20 carbon atoms.

22. A cosmetic process for treating a keratin materials material, the process comprising: applying the composition of claim 1 to the keratin material.

23. A cosmetic treatment process for hiding skin color imperfections and/or skin relief imperfections, the process comprising: applying the composition of claim 1 to the skin.

24. An aqueous dispersion composition, comprising at least one composition of claim 1 in an aqueous phase.
